(19) United States
(12) Patent Application Publication
(10) Pub. No.: US 2024/0425911 A1
(43) Pub. Date: Dec. 26, 2024

(54) PROTRACTOR ISOTHERMAL NUCLEIC ACID DETECTION METHODS

(71) Applicant: SHANGHAI JIAO TONG UNIVERSITY, Shanghai (CN)

(72) Inventors: Litao YANG, Shanghai (CN); Yongkun GUO, Shanghai (CN)

(73) Assignee: SHANGHAI JIAO TONG UNIVERSITY, Shanghai (CN)

(21) Appl. No.: 18/800,086

(22) Filed: Aug. 11, 2024

Related U.S. Application Data
(63) Continuation of application No. PCT/CN2022/140597, filed on Dec. 21, 2022.

(30) Foreign Application Priority Data
Jun. 21, 2022 (CN) 202210703566.5

(51) Int. Cl. C12Q 1/6851; C12Q 1/70

(52) U.S. Cl. CPC C12Q 1/6851; C12Q 1/701

(57) ABSTRACT

The present disclosure provides a PROTRACTOR isothermal nucleic acid detection method, comprising: S1, extracting total nucleic acid from a sample to be detected; S2, configuring a reaction system, the reaction system including a single-stranded DNA (ssDNA) probe, an RNA fluorescent probe, a DNA ligase or a variant thereof, an RNA polymerase or a variant thereof, a guide RNA or a derivative thereof, a CRISPR-related Cas protein or a variant thereof, and a PROTRACTOR reaction buffer; wherein the ssDNA probe is specifically complementary to a strand of a target nucleic acid molecule; S3, adding the total nucleic acid extracted in the step S1 to the reaction system of the step S2 to perform thermostatic reaction and generating a fluorescent signal; wherein the ssDNA probe forms a single-stranded circular DNA probe under the action of the DNA ligase or the variant thereof in a process of thermostatic reaction; and S4, reading and recording the fluorescent signal generated in the step S3, and determining presence or absence of the target nucleic acid molecule in the sample to be detected by the fluorescent signal.

### CROSS-REFERENCE TO RELATED APPLICATION

This application is a Continuation of International Application No. PCT/CN2022/140597, filed on Dec. 21, 2022, which claims priority to Chinese Patent Application No. 202210703566.5, filed on Jun. 21, 2022, the entire contents of each of which are hereby incorporated by reference.

### SEQUENCE LISTING

The instant application contains a Sequence Listing which has been submitted electronically in XML format and is hereby incorporated by reference in its entirety. The XML copy, created on Sep. 2, 2024, is named “2024 Sep. 2-Sequence Listing-65617-H023US00,” and is 19,739 bytes in size.

### TECHNICAL FIELD

The present disclosure relates to a field of rapid nucleic acid detection in molecular biology, in particular to a PROTRACTOR isothermal nucleic acid detection method, and relates to a new integrated detection technology of nucleic acid isothermal amplification and signal output, which realizes a rapid, one-step, single-tube amplification and detection of specific DNA or RNA by utilizing a DNA ligase, an RNA polymerase, and a CRISPR/Cas protein.

### BACKGROUND

Quantitative polymerase chain reaction (qPCR) is the current gold standard for molecular detection of nucleic acid. Different from deoxyribonucleic acid (DNA) detection, in vitro detection of ribonucleic acid (RNA) often requires a two-step process: (1) reverse transcription of the RNA into cDNA under the action of a reverse transcriptase; and (2) qPCR amplification and detection analysis of a cDNA sample. Although qPCR is a well-established technology and widely used in the medical field, it still requires large and expensive thermal cyclers, sample transportation and preservation, professional operators, and long detection time (at least 2-4 h from a sample to a result), which largely limits the use of qPCR in a resource limited environment and point-of-care (POC) analysis.

Nucleic acid isothermal amplification technology (IAT) has emerged as a promising alternative for rapid and efficient amplification of target nucleic acid molecules at a thermostatic condition without a thermal cycler required for qPCR. In addition, isothermal amplification may be carried out under simple conditions, such as a room temperature, a water bath, etc., especially useful for rapid detection of nucleic acid molecules in resource limited regions, which may not be achieved by qPCR. Dozens of isothermal nucleic acid amplification techniques have been developed, such as loop-mediated isothermal amplification (LAMP), which is a technique that uses 4-6 pairs of primers to recognize target-specific sites and uses the DNA polymerase with strand displacement activity at 60-65° C. to achieve efficient (within 1 h) amplification detection of nucleic acids. Recombinase polymerase amplification (RPA) refers to a technology which simulates the in vivo nucleic acid replication mechanism at a thermostatic condition of 37-42° C., where various key enzymes or proteins assist in the amplification of the DNA polymerase to achieve the exponential amplification of nucleic acids. The entire reaction usually takes 20-30 min. Nucleic acid sequence-based amplification (NASBA) achieves rapid and continuous amplification of RNA at about 41° C. (about 60 min) by utilizing reverse transcriptase, T7 RNA polymerase, RNase H, and two oligonucleotide primers. Rolling circle amplification (RCA) refers to a technology which utilizes DNA ligase and DNA polymerase with strand replacement activity to perform strand displacement of circular templates under the guidance of one or more primers at 30° C. to produce a plurality of long single strands with target sequences. The entire process usually takes 2 h.

However, the above several isothermal amplification techniques all have their own shortcomings. For example, 4-6 primers are required for LAMP, the design requirements for the primers are relatively high, and the detection of point mutations or modification sites often fails to meet the requirements. The product after LAMP is very liable to form aerosol contamination when the lid is opened, causing false positive results. The enzyme component of RPA is relatively complex, has relatively high requirements for buffer solution, and the stability of the reaction system is low, which is easy to cause poor repeatability. In addition, the mutation sites cannot be detected. Meanwhile, LAMP and RPA still need reverse transcription for RNA sample detection. NASBA is not suitable for DNA detection, and the reaction components are relatively complex and the stability is poor, susceptible to the influence of matrix. The reaction time of RCA is relatively long, and the advantage in on-site detection is not obvious. Rolling circle transcription (RCT) is a kind of isothermal amplification reaction catalyzed by RNA polymerase with in vitro transcription activity. After one strand of the target nucleic acid molecule hybridizes with the single-stranded DNA probe, it is connected to form a circular template under the action of the DNA ligase, and is continuously transcribed along the circular template into a repetitive long single-stranded RNA product containing the target sequence under the action of RNA polymerase, achieving efficient amplification at the room temperature. However, there is a lack of effective and specific method for detecting the product of amplified single-stranded RNA.

The clustered regularly interspaced short palindromic repeat (CRISPR) system is an immune system that bacteria or archaea have evolved to defend themselves against viral infections by recognizing foreign genetic material and integrating it into the CRISPR sequence of their own genomes, and then accurately shearing the exogenous nucleic acids by Cas nuclease when the foreign genetic material reinvades them. Cas nuclease is an important related protein in CRISPR. Cas13a is a newly identified CRISPR nuclease in recent years. This nuclease has the ability to be activated by specific RNA to obtain non-specific RNA nuclease activity, thus shearing other single-stranded RNA. Cas13a together with RNA fluorescence reporter system may detect specific RNA products. However, Cas13a has a low sensitivity when acting alone, and may only achieve the detection of nucleic acids at the level of fM to pM, and the sensitivity for molecular diagnosis is poor, such as direct detection of potential tumor markers miR-19b and miR-20a without amplification of nucleic acid, with a detection limit of 10 pM. In 2017 and 2019, Feng Zhang et al., developed SHERLOCK, a new method for detecting a nucleic acid by using Cas13a protein combined with the isothermal amplification technique RPA, and the sensitivity may realize the detection of aM-level samples. However, the SHERLOCK is not able to realize one-step single-tube detection for RNA, and RPA is complicated in composition and poor in stability, which increases the difficulty of operation.

Therefore, it is desirable to develop a simple, stable, and reliable nucleic acid assay based on the IAT for single-tube nucleic acid detection.

### SUMMARY

One or more embodiments of the present disclosure provide a PROTRACTOR isothermal nucleic acid detection method, comprising:
S1, extracting total nucleic acid from a sample to be detected;
S2, configuring a reaction system, the reaction system including a single-stranded DNA (ssDNA) probe, an RNA fluorescent probe, a DNA ligase or a variant thereof, an RNA polymerase or a variant thereof, a guide RNA or a derivative thereof, a CRISPR-related Cas protein or a variant thereof, and a PROTRACTOR reaction buffer, wherein the ssDNA probe may be specifically complementary to a strand of a target nucleic acid molecule;
S3, adding the total nucleic acid extracted in the step S1 to the reaction system of the step S2 to perform thermostatic reaction and generating a fluorescent signal, the ssDNA probe forming a single-stranded circular DNA probe under the action of the DNA ligase or the variant thereof in a process of thermostatic reaction; and
S4, reading and recording the fluorescent signal generated in the step S3, and determining presence or absence of the target nucleic acid molecule in the sample to be detected by the fluorescent signal.

In some embodiments, the PROTRACTOR may be a universal nucleic acid detection platform capable of detecting different types of nucleic acid molecules, including one or more of ssDNA, double-stranded DNA (dsDNA), and single-stranded RNA (ssRNA).

In some embodiments, the CRISPR-related Cas protein or the variant thereof may be a nuclease having an ssRNA recognition shear function and a trans-single-stranded RNA shear function. The CRISPR-related Cas protein or the variant thereof may include any one of LbaCas13, LbuC13a, LwaCas13a, AspCas13b, BzoCas13b, CcaCas13b, PsmCas13b, PinCas13b, Pin2Cas13b, Pin3Cas13b, PbuCas13b, PguCas13b, PigCas13b, PsaCas13b, RanCas13b, PspCas13b, EsCas13d, and RspCas13d, or any variant thereof.

In some embodiments, the ssDNA probe may be composed of a 5′ end arm, a linking sequence, and a 3′ end arm in series. Sequences of the 5′ end arm and the 3′ end arm may be complementary to one strand of a target nucleic acid molecule sequence. The linking sequence may be a DNA sequence including a complementary sequence of a T7 promoter (T7p).

In some embodiments, the RNA fluorescent probe may be an ssRNA with a 5′ end labeled with any fluorescence group of FAM, HEX, VIC, Cy5, Cy3, TET, ROX, FITC, and Joe, and a 3′ end labeled with any fluorescence quenching group of TAMRA, BHQ1, MGB, and BHQ2.

In some embodiments, the DNA ligase may catalyze formation of a phosphodiester bond between two nucleotide strands using the energy of ATP, and may be used to join dsDNA molecules or ssDNA gaps of an RNA/DNA hybrid duplex. The DNA ligase or the variant thereof may include any one of a T4 DNA ligase, an E. coli DNA ligase, a SplintR ligase, and a HiFi Taq DNA ligase, or any variant thereof.

In some embodiments, the guide RNA or the derivative thereof may be complementary to a sequence of the target nucleic acid molecule.

In some embodiments, main components of the PROTRACTOR reaction buffer may include 0.1 mM-5 mM NTPs, 10 mM-100 mM Tris-HCl, 0.5 mM-10 mM MgCl₂, 0.01 mM-10 mM ATP, and 0.5 mM-10 mM DTT, pH of the PROTRACTOR reaction buffer being within a range of 6.5-8.0.

In some embodiments, the RNA polymerase or the variant thereof may be selected from any one of a T7 RNA polymerase, an E. coli RNA polymerase, a T3 RNA polymerase, and an SP6 RNA polymerase, or any variant thereof.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering indicates the same structure, wherein:

FIG. 1 is a schematic diagram illustrating a process for detecting ssDNA or RNA samples according to some embodiments of the present disclosure;

FIG. 2 is a schematic diagram illustrating a process for detecting a dsDNA sample according to some embodiments of the present disclosure;

FIG. 3 is a schematic diagram illustrating a fluorescent signal detection result of RNA, dsDNA, and ssDNA molecules detected by a PROTRACTOR isothermal nucleic acid detection method according to some embodiments of the present disclosure;

FIG. 4 is a schematic diagram illustrating a sensitivity result of an ssRNA sample detected by a PROTRACTOR isothermal nucleic acid detection method according to some embodiments of the present disclosure;

FIG. 5 is a schematic diagram illustrating a fluorescent signal detection result of an N gene in SARS-COV-2 detected by a PROTRACTOR isothermal nucleic acid detection method according to some embodiments of the present disclosure;

FIG. 6 is a schematic diagram illustrating a fluorescent signal detection result for differentiating between SARS-COV-2 subtypes using a PROTRACTOR isothermal nucleic acid detection method according to some embodiments of the present disclosure; and

FIG. 7 is a schematic diagram illustrating comparison of RNA detection results with different buffers according to some embodiments of the present disclosure.

### DETAILED DESCRIPTION

In order to more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for a person having ordinary skills in the art to apply the present disclosure to other similar scenarios in accordance with these drawings without creative labor. Unless obviously obtained from the context or the context illustrates otherwise, the same numeral in the drawings refers to the same structure or operation.

It should be understood that “system”, “device”, “unit” and/or “module” as used herein is a method for distinguishing different components, elements, parts, portions or assemblies of different levels. However, the words may be replaced by other expressions if other words can achieve the same purpose.

As indicated in the disclosure and claims, the terms “a”, “an”, and/or “the” are not specific to the singular form and may include the plural form unless the context clearly indicates an exception. Generally speaking, the terms “comprising” and “including” only suggest the inclusion of clearly identified steps and elements, and these steps and elements do not constitute an exclusive list, and the method or device may also contain other steps or elements.

The flowchart is used in the present disclosure to illustrate the operations performed by the system according to the embodiments of the present disclosure. It should be understood that the preceding or following operations are not necessarily performed in the exact order. Instead, various steps may be processed in reverse order or simultaneously. Meanwhile, other operations may be added to these procedures, or a certain step or steps may be removed from these procedures.

The embodiments of the present disclosure provide a PROTRACTOR isothermal nucleic acid detection method, particularly provides a novel method (PROTRACTOR) for nucleic acid detection based on an ssDNA cyclization, transcription, and a CRISPR-related Cas protein or a variant thereof. The cyclization of an ssDNA probe may be achieved using a DNA ligase, converting a target molecule signal into a circular DNA signal. Efficient transcription of an RNA polymerase may be completed using the circular DNA as a template. The CRISPR-related Cas protein or the variant thereof may recognize and shear a transcription product under the guidance of crRNA, and carries out the detection reaction while completing the amplification. A reaction buffer system may be optimized such that ligation, amplification and detection reactions may be carried out in the same reaction tube, realizing ultra-sensitive, highly specific and rapid detection of a target nucleic acid molecule (RNA, ssDNA, or dsDNA). The method may be free from limitations of DNA amplification and primers in conventional nucleic acid detection. When RNA sample detection is performed, no reverse transcription may be required, and amplification detection of the RNA molecule may be directly carried out.

The embodiments of the present disclosure provide a PROTRACTOR isothermal nucleic acid detection method, comprising:
S1, extracting total nucleic acid from a sample to be detected;
S2, configuring a reaction system, the reaction system including a single-stranded DNA (ssDNA) probe, an RNA fluorescent probe, a DNA ligase or a variant thereof, an RNA polymerase or a variant thereof, a guide RNA or a derivative thereof, a CRISPR-related Cas protein or a variant thereof, and a PROTRACTOR reaction buffer, the ssDNA probe being specifically complementary to a strand of a target nucleic acid molecule;
S3, adding the total nucleic acid extracted in the step S1 to a reaction system of the step S2 to perform thermostatic reaction and generating a fluorescent signal, wherein the ssDNA probe may form a single-stranded circular DNA probe under the action of the DNA ligase or the variant thereof in a process of thermostatic reaction; and
S4, reading and recording the fluorescent signal generated in the step S3, and determining presence or absence of the target nucleic acid molecule in the sample to be detected by the fluorescent signal.

In some embodiments, the ssDNA probe may be composed a DNA sequence complementary to one strand of a target nucleic acid molecule sequence and a T7p-containing linking sequence.

The technical principle of the invention is as follows:
the ssDNA probe sequence may be specifically complementary to one strand of the target nucleic acid molecule, and the ssDNA probe may form the single-stranded circular DNA probe under the action of the DNA ligase or the variant thereof;
the ssDNA probe may be configured to specifically recognize and bind to a target sequence on one strand of the target nucleic acid molecule, and may be used as a template for amplification after cyclization. The ssDNA probe may be composed of the DNA sequence complementary to one strand of the target nucleic acid molecule sequence and the linking sequence. The linking sequence may contain a T7 promoter complementary sequence (T7p) capable of being recognized and bound by the RNA polymerase to initiate transcription;
when sample detection is performed, the single-stranded circular DNA probe may be continuously transcribed into long single-stranded RNA containing a plurality of repeats of the target sequence of the target nucleic acid molecule in the presence of the RNA polymerase or the variant thereof;
a large number of ssRNA amplicons may be formed by transcription and recognized by binary complexes formed by the guide RNA or the derivative thereof and the CRISPR-related Cas protein or the variant thereof, and the RNA fluorescent probe may be sheared to generate a detectable fluorescent signal; and
the fluorescent signal generated by the PROTRACTOR may be read and recorded using a fluorescence detector to determine presence or absence of the target nucleic acid molecule in the sample to be detected.

If the target nucleic acid molecule is dsDNA, pre-denaturation of dsDNA may be performed prior to the reaction.

In some embodiments, the PROTRACTOR may be a universal nucleic acid detection platform capable of detecting different types of nucleic acid molecules. In some embodiments, the PROTRACTOR may detect one or more of the ssDNA, the dsDNA, and the ssRNA.

In some embodiments, the sample to be detected may be a biological sample. In some embodiments, the sample to be detected may be a tissue, a cell, or the like. In some embodiments, the sample to be detected may be an organism such as a virus, a bacterium, a fungus, a plant or animal, or the like.

The total nucleic acid refers to a sum of different types of nucleic acids present in the sample to be detected. In some embodiments, the total nucleic acid may include DNA and RNA. In some embodiments, the total nucleic acid may include the ssDNA, the ssRNA, and the dsDNA.

The reaction system refers to a reaction mixture for nucleic acid amplification. In some embodiments, the reaction system may include a probe, an enzyme, a primer, a buffer, or the like. The probe refers to a small fragment of nucleic acid used to detect a nucleic acid sequence complementary to the probe. In some embodiments, the probe may be an ssDNA probe. In some embodiments, the probe may be an ssRNA probe. In some embodiments, the enzymes in the reaction system of nucleic acid amplification may include any one or more enzymes capable of catalyzing nucleic acid amplification. In some embodiments, the enzyme in the reaction system of nucleic acid amplification may include the DNA ligase or the variant thereof, and the RNA polymerase or the variant thereof.

In some embodiments, the reaction system may include the ssDNA probe, the RNA fluorescent probe, the DNA ligase or the variant thereof, the RNA polymerase or the variant thereof, the guide RNA or the derivative thereof, the CRISPR-related Cas protein or the variant thereof, and the PROTRACTOR reaction buffer.

In some embodiments, the ssDNA probe may be composed of a 5′ end arm, a linking sequence, and a 3′ end arm in series. The sequences of the 5′ end arm and the 3′ end arm may be complementary to one strand of the target nucleic acid molecule sequence. The linking sequence may be a DNA sequence including a complementary sequence of a T7 promoter (T7p). The T7 promoter is derived from a T7 phage and is a segment of sequence that initiates transcription of a T7 phage gene. The T7 promoter refers to a class of strong promoters.

The RNA fluorescent probe refers a probe labeled with a fluorescence group or a fluorescence quenching group at the 5′ end and/or the 3′ end of the RNA probe. In some embodiments, the RNA fluorescent probe may be an ssRNA with a 5′ end labeled with any fluorescence group of FAM, HEX, VIC, Cy5, Cy3, TET, ROX, FITC, and Joe, and a 3′ end labeled with any fluorescence quenching group of TAMRA, BHQ1, MGB, and BHQ2.

As used herein, the fluorescence group refers to a group capable of emitting the fluorescent signal, and the fluorescence quenching group may absorb the fluorescent signal emitted by the fluorescence group to inhibit the emission of the fluorescent signal.

The DNA ligase refers to a ligase capable of catalyzing joining of ends of two DNA strands. In some embodiments, the DNA ligase may catalyze formation of a phosphodiester bond between two nucleotide strands using the energy of ATP, and may be used to join dsDNA molecules or ssDNA gaps of an RNA/DNA hybrid duplex. In some embodiments, the DNA ligase or the variant thereof may include any one of a T4 DNA ligase, an E. coli DNA ligase, a SplintR ligase, and a HiFi Taq DNA ligase or any variant thereof. The DNA ligase may include a mixture of one and/or more wild-type, modified, codon-optimized, evolved, thermophilic, chimeric, engineered DNA ligase. In some embodiments, the DNA ligase may be preferably the T4 DNA ligase.

In some embodiments, the DNA ligase is capable of specifically ligating the phosphodiester bond of the ssDNA probe that hybridizes to one strand of the target nucleic acid molecule sequence to form a circular DNA template.

The RNA polymerase refers to an enzyme that catalyzes RNA synthesis. In some embodiments, the RNA polymerase may catalyze RNA synthesis using the DNA as a template. In some embodiments, the RNA polymerase may catalyze RNA synthesis using the RNA as a template. In some embodiments, the RNA polymerase may be selected from any one of a T7 RNA polymerase, an E. coli RNA polymerase, a T3 RNA polymerase, an SP6 RNA polymerase. In some embodiments, the RNA polymerase may be preferably the T7 RNA polymerase. In some embodiments, the RNA polymerase may include a mixture of one and/or more of wild-type, modified, codon-optimized, evolved, thermophilic, chimeric, engineered reverse transcriptase.

In some embodiments, the RNA polymerase may recognize and bind to a T7p zone of the circular DNA template to initiate efficient transcription and generate a large number of repeat long ssRNA products containing the target sequence.

The guide RNA refers to a short RNA sequence that directs a Cas protein to shear a specific site of a nucleic acid. In some embodiments, the guide RNA may bind to the Cas protein. In some embodiments, the guide RNA may bind to the target nucleic acid molecule.

In some embodiments, the guide RNA or the derivative thereof may be complementary to the sequence of the target nucleic acid molecule.

In some embodiments, the CRISPR-related Cas protein or the variant thereof may be a nuclease having an ssRNA recognition and shear function and a trans-ssRNA shear function. In some embodiments, the CRISPR-related Cas protein or the variant thereof may include a mixture of one and/or more of wild-type, modified, codon-optimized, evolved, thermophilic, chimeric, and engineered Cas protein. In some embodiments, the CRISPR-related Cas protein or the variant thereof may include LbaCas13, LbuC13a, LwaCas 13a, AspCas13b, BzoCas13b, CcaCas13b, PsmCas13b, PinCas13b, Pin2Cas13b, Pin3Cas13b, PbuCas13b, PguCas13b, PigCas13b, PsaCas13b, RanCas13b, PspCas13b, EsCas13d, and RspCas13d, or any variant thereof. In some embodiments, the CRISPR-related Cas protein or the variant thereof may be preferably Cas13a.

In some embodiments, the CRISPR-related Cas protein or the variant thereof and the guide RNA may form a binary complex, which in turn may bind specifically to a target RNA sequence to form a ternary complex, thereby activating the non-specific RNA nuclease activity to shear the ssRNA molecule in the reaction system.

In some embodiments, main components of the PROTRACTOR reaction buffer may include 0.1 mM-5 mM NTPs, 10 mM-100 mM Tris-HCl, 0.5 mM-10 mM MgCl₂, 0.01 mM-10 mM ATP, and 0.5 mM-10 mM DTT. In some embodiments, pH of the PROTRACTOR reaction buffer may be within a range of 6.5-8.0.

In some embodiments, in order to shorten the reaction time of the PROTRACTOR, improve the detection efficiency, and make it more suitable for rapid on-site detection, especially for use in resource limited regions, specific composition and concentration of the PROTRACTOR reaction buffer may be optimized. In some embodiments, the PROTRACTOR reaction buffer may be optimized to be 0.1 mM-5 mM NTPs, 10 mM-50 mM Tris-HCl, 1 mM-10 mM MgCl₂, 0.01 mM-10 mM ATP, and 0.5 mM-5 mMDTT, and pH of the PROTRACTOR reaction buffer may be within a range of 7.0-8.0.

In some embodiments, the PROTRACTOR reaction buffer may include 0.1 mM-5 mM NTPs, 40 mM Tris-HCl, 10 mM MgCl₂, 0.01 mM-10 mM ATP, and 1 mM DTT, and pH of the PROTRACTOR reaction buffer may be within a range of 7.0-8.0. The reaction system was composed of 0.1 pM-4 PM FAM and BHQ1 dual-labeled RNA fluorescent probe, 0.1 pM-5 pM guide RNA, and an enzyme mixture (1 U-20 U T4 DNA ligase; 10 U-100 U T7 RNA polymerase; 0.01 pM-5 pM LwaCas13a protein).

The embodiments of the present disclosure further provide a reaction system for nucleic acid detection, comprising a ssDNA probe, an RNA fluorescent probe, a DNA ligase or a variant thereof, an RNA polymerase or a variant thereof, a guide RNA or a derivative thereof, a CRISPR-related Cas protein or a variant thereof, and a PROTRACTOR reaction buffer.

A kit comprising the reaction system also fall within the scope of protection of the present disclosure. The kit for nucleic acid detection can achieve precise, rapid and highly sensitive detection of a specific target nucleic acid molecule (RNA, ssDNA, or dsDNA) under the isothermal condition at room temperature.

The embodiments of the present disclosure can rapidly complete the detection of DNA or RNA molecules under the isothermal condition at room temperature. Firstly, the nucleic acid of a sample to be detected may be obtained by rapid nucleic acid extraction; and then a combination of the ligase, the transcriptase, and CRISPR-related protein, the ssDNA probe, and the nucleic acid fluorescent probe may react with the nucleic acid to be detected under the isothermal condition, and finally, the presence of the target nucleic acid molecule in the sample to be detected may be determined by the detection of the fluorescent signal.

The method and the kit of the embodiments of the present disclosure are a universal and versatile rapid detection platform for detecting nucleic acid molecules of viruses, bacteria, fungi, plants and animals, and other organisms.

Beneficial effects that may be achieved by the embodiments of the present disclosure include, but are not limited to, the following content:
1. high sensitivity: detection of a single molecule (single copy) can be realized by detecting the target nucleic acid molecule (ssDNA, dsDNA, and RNA) by using the method of the present disclosure;
2. Universality: detection of the DNA or the RNA can be realized by using the method of the present disclosure, and point mutations can be distinguished;
3. Composition stability: the method of the present disclosure does not require DNA amplification or primers, and does not require reverse transcription for detection of the RNA sample;
4. Rapid: the method of the present disclosure can complete the detection within 10 min;
5. Convenient: the method of the present disclosure realizes an isothermal reaction of a single buffer in a single tube, which is convenient to operate and easy to follow steps, and is suitable for rapid detection of the nucleic acid molecules in resource limited regions;
6. low false positive: the ssDNA probe of the present disclosure is cyclized after the ssDNA probe specifically binds to one strand of the target nucleic acid molecule to trigger the amplification reaction. The shear reaction is activated to generate the fluorescent signal only after the amplicon is recognized by the binary complex formed by the specific guide RNA and the CRISPR-related Cas protein or the variant thereof, overcoming the problem of false positive from the reaction mechanism. In addition, the amplification product in the present disclosure is the RNA rather than the DNA, and the RNA is easy to degrade and not easy to produce aerosols, making the detection method overcome the problem of easy contamination of LAMP and qPCR. Meanwhile, the method of the present disclosure is a closed tube reaction, which is physically isolated, minimizing the possibility of contamination;
7. isothermal detection: the three engineered enzymes including the DNA ligase or the variant thereof, the RNA polymerase or the variant thereof, and the CRISPR-related Cas protein or the variant thereof and a variety of chemical components together create an environment that maximizes simulation of the amplification of nucleic acids in organisms, and each engineered enzyme performs its own function and works at the optimum reaction temperature, such that the highest working efficiency is realized; and
8. one-step method: in order to make the operation easier, reduce the contamination caused by opening the lid and adding samples, and improve the reaction efficiency and shorten the reaction time to be more suitable for on-site rapid detection, especially for use in the resource limited regions, ssDNA probe cyclization, transcription, and shearing of the CRISPR-related Cas protein or the variant thereof are innovatively integrated into the same reaction tube, realizing the one-step method.

The present disclosure is described in detail below with reference to Examples. The following Examples are helpful to those skilled in the art to further understand the present disclosure, but do not limit the present disclosure in any way. It should be noted that to those having ordinary skill in the art, a number of adjustments and improvements may be made without departing from the conception of the present disclosure. Such adjustments and improvements fall within the scope of protection of the present disclosure.

### EXAMPLES

### Example 1 a Method for Detecting a dsDNA Target

A dsDNA (Target 1) was selected as a target sequence. The Target 1 sequence is shown in SEQ ID NO. 1:

(SEQ ID NO. 1)
GTGATGAAGTCAGACAAATCGCTCCAGGGCAAACTGGAAAGATTGCTGA
TTATAATTATAAATTACCAGATGATTTTACAGGCTGCGTTATAGCTTGG
AATTCTAACAATCTTGATTCTAAGGTTGGTGGTAATTATAATTA
CCTGTATAGATTGTTTAGGAAGTCTAATCTCAAACCTTTTGAGAGAGAT
ATTTCAACTGAAATCTATCAGGCCGGTAGCACACCTTGTAATGGTGTTG
AAGGT;

preparation of a guide RNA: synthesizing a primer containing a T7 promoter sequence. A reverse primer (T7p-R) for a complementary sequence of the T7 promoter: crRNA-target-R is shown in SEQ ID NO. 2:
   TTCTTAATACGACTCACTATAGGGATTTAGACTACCCCAAAAACGAAGGGGACTAA AACGGTAATTATAATTACCACCAACCT (SEQ ID NO. 2), and a forward primer (T7p-F) for the complementary sequence of the T7 promoter: crRNA-target-F is shown in SEQ ID NO. 3: CCTATAGTGAGTCGTATTAAGAA (SEQ ID NO. 3). Incomplete double-stranded crDNA was made by double-primer annealing and used as a template for in vitro transcription of the guide RNA. A T7 transcriptase was used for catalyzing transcription to perform an overnight reaction at 37° C. The crRNA was obtained by using a RNA Clean& Concentrator 100 kit for purification and stored at −20° C. or −80° C.

A ssDNA probe sequence of the Target 1 (padlock1) is shown in SEQ ID NO. 4:

(SEQ ID NO. 4)
TACCACCAACCTCCAACCTAAACCCTATAGTGAGTCGTATTAATCCCGC
CTACAGGTAATTATAAT;

As shown in FIG. 2, the PROTRACTOR isothermal nucleic acid detection method includes the following steps:
(1) dsDNA was extracted from a sample to be detected;
(2) a ssDNA probe was mixed with the dsDNA extracted in the step (1), annealed at high temperature (80° C.-95° C.) for 5 min, and then naturally cooled to room temperature and added to a reaction system, wherein the reaction system was composed of 0.8 pM FAM and BHQ1 dual-labeled RNA fluorescent probe, 0.5 pM guide RNA, an enzyme mixture (10 U T4 DNA ligase; 10 U T7 RNA polymerase; 0.5 pM LwCas 13a protein), and a PROTRACTOR reaction buffer, the RNA fluorescent probe is 6-FAM-mArArUrGrGrCmAmArArUrGrGrCmA-BHQ1, the PROTRACTOR reaction buffer included 0.5 mM NTPs, 40 mM Tris-HCl, 10 mM MgCl₂, 0.01 mM-10 mM ATP, and 1 mM DTT, and pH of the PROTRACTOR reaction buffer is within a range of 7.0-8.0;
(3) fluorescence detection: after the reaction system was mixed, the temperature was set at 37° C. in a fluorescence real-time quantitative PCR instrument (7900 HT Fast Real-Time PCR), wherein a fluorescence group of the dual-labeled RNA fluorescent probe was FAM, an acquisition interval of a fluorescent signal was 1 min, and the detection time was 30 min.

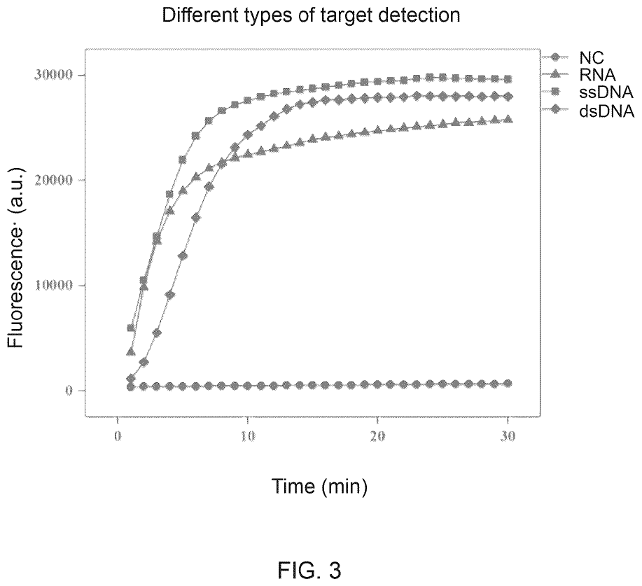

The result of fluorescence detection is shown in FIG. 3. The dsDNA can be detected by the method.

### Example 2 a Method for Detecting an ssDNA Target

A ssDNA (Target 2) was selected as a target sequence. The Target 2 sequence is shown in SEQ ID NO. 5:

GATTCTAAGGTTGGTGGGTAATTATAATTACCTGTATAGATTGTTTAGGAAGTCTAA TC TCA (SEQ ID NO. 5). The Target 2 sequence was synthesized by Shanghai Sangon. The synthesized sequence was dissolved in enzyme-free sterile water and diluted to 10 μM;

Preparation of a guide RNA: a primer containing a T7 promoter sequence was synthesized. A reverse primer (T7p-R) for a complementary sequence of the T7 promoter: crRNA-target-R is shown in SEQ ID NO. 2; a forward primer (T7p-F) for the complementary sequence of the T7 promoter: crRNA-target-F is shown in SEQ ID NO. 3. Incomplete double-stranded crDNA was made by double-primer annealing and used as a template for in vitro transcription of the guide RNA. A T7 transcriptase was used for catalyzing transcription, and then subjected to an overnight reaction at 37° C. The crRNA was obtained by using an RNA Clean& Concentrator 100 kit for purification and stored at −20° C. or −80° C.

A ssDNA probe sequence (padlock2) of the Target 2 is shown in SEQ ID NO. 4.

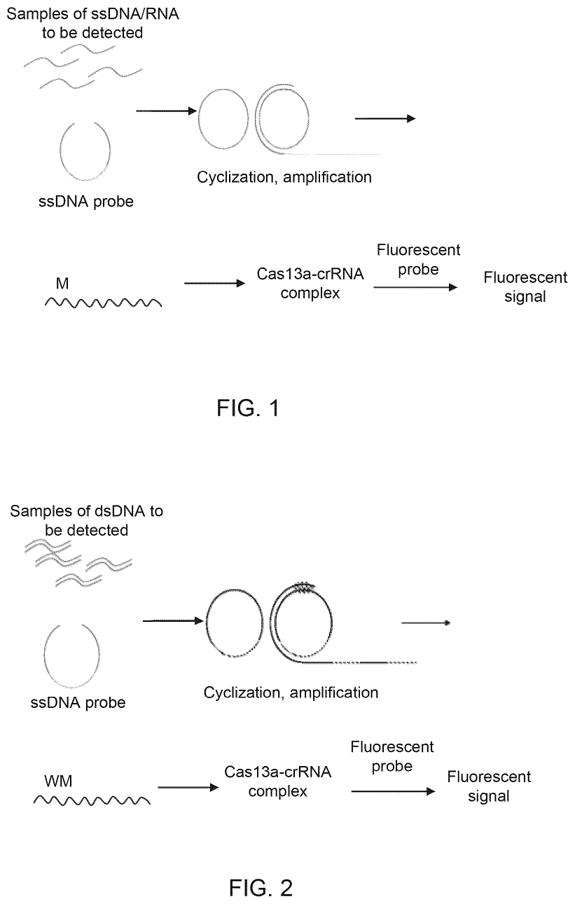

As shown in FIG. 1, the PROTRACTOR isothermal nucleic acid detection method comprises the following steps. Firstly, an ssDNA probe and target ssDNA were added to a reaction system. The reaction system was composed of 0.8 pM FAM and BHQ1 dual-labeled RNA fluorescent probe, 0.5 pM guide RNA, an enzyme mixture (10 U T4 DNA ligase; 10 U T7 RNA polymerase; 0.5 pM LwaCas13a protein), and a PROTRACTOR reaction buffer. The RNA fluorescent probe was 6-FAM-mArArUrGrGrCmAmArArUrGrGrCmA-BHQ1. The PROTRACTOR reaction buffer included 0.5 mM NTPs, 40 mM Tris-HCl, 10 mM MgCl₂, 0.01 mM-10 mM ATP, and 1 mM DTT, and pH of the PROTRACTOR reaction buffer was within a range of 7.0-8.0.

Fluorescence detection: after mixing of reaction, the temperature was set at 37° C. in a fluorescence real-time quantitative PCR instrument (7900HT Fast Real-Time PCR), a fluorescence group of a dual-labeled RNA fluorescent probe was FAM, an acquisition interval of a fluorescent signal was 1 min, and the detection time was 30 min.

The result of fluorescence detection is shown in FIG. 3. The ssDNA can be detected by the method.

### Example 3 a Method for Detecting an RNA Target

An RNA (Target 3) was selected as a target sequence. The Target 3 sequence is shown in SEQ ID NO. 6:

(SEQ ID NO. 6)
GUGAUGAAGUCAGACAAAUCGCUCCAGGGCAAACUGGAAAGAUUGCUGA
UUAUAAUUAUAAAUUACCAGAUGAUUUUACAGGCUGCGUUAUAGCUUGG
AAUUCUAACAAUCUUGAUUCUAAGGUUGGUGGUAAUUAUAAUUACCUGU
AUAGAUUGUUUAGGAAGUCUAAUCUCAAACCUUUUGAGAGAGAUAUUUC
AACUGAAAUCUAUCAGGCCGGUAGCACACCUUGUAAUGGUGUUGAAGG
U;

preparation of a guide RNA: a primer containing a T7 promoter sequence was synthesized. A reverse primer (T7p-R) for a complementary sequence of the T7 promoter: crRNA-target-R is shown in SEQ ID NO. 2, and a forward primer (T7p-F) for the complementary sequence of the T7 promoter: crRNA-target-F is shown in SEQ ID NO. 3. Incomplete double-stranded crDNA was made by double-primer annealing and used as a template for in vitro transcription of the guide RNA. A T7 transcriptase was used for catalyzing transcription, and then subjected to an overnight reaction at 37° C. The crRNA was obtained by using a RNA Clean& Concentrator 100 kit for purification and stored at −20° C. or −80° C.

A ssDNA probe sequence (padlock3) of the Target 3 is shown in SEQ ID NO. 4.

The PROTRACTOR isothermal nucleic acid detection method comprises the following steps. Firstly, an ssDNA probe and Target 3 RNA were added to a reaction system. The reaction system was composed of 0.8 pM FAM and BHQ1 dual-labeled RNA fluorescent probe, 0.5 pM guide RNA, an enzyme mixture (10 U T4 DNA ligase; 10 U T7 RNA polymerase; 0.5 pM LwaCas13a protein), and a PROTRACTOR reaction buffer. The RNA fluorescent probe was 6-FAM-mArArUrrGrGrCmAmArArUrGrGrCmA-BHQ1. The PROTRACTOR reaction buffer included 0.5 mM NTPs, 40 mM Tris-HCl, 10 mM MgCl₂, 0.01 mM-10 mM ATP, and 1 mM DTT, and pH of the PROTRACTOR reaction buffer was within a range of 7.0-8.0.

Fluorescence detection: after mixing of the reaction, the temperature was set at 37° C. in a fluorescence real-time quantitative PCR instrument (7900HT Fast Real-Time PCR), a fluorescence group of the dual-labeled RNA fluorescent probe was FAM, an acquisition interval of a fluorescent signal was 1 min, and the detection time was 30 min.

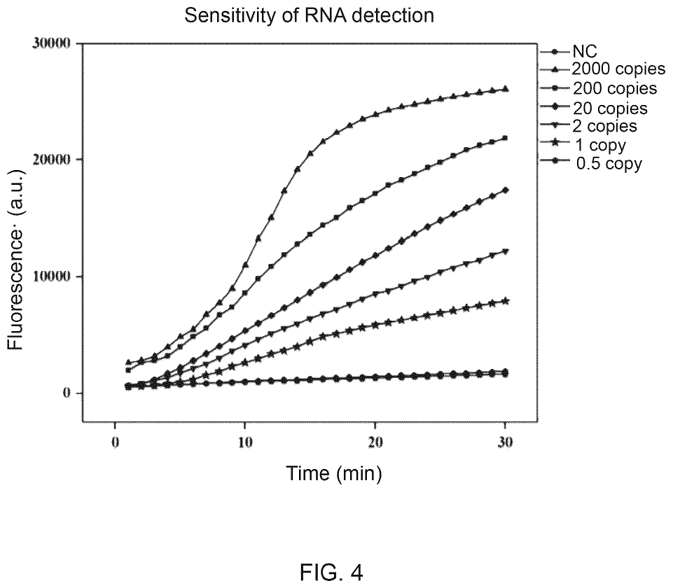

The result of fluorescence detection is shown in FIG. 3. The ssRNA can be detected by the method. As shown in FIG. 4, the ssRNA molecules as low as 1 copy per reaction can be detected by the method.

### Example 4 a Method for Detecting SARS-COV-2

SARS-COV-2 is an RNA virus. Nasopharyngeal swab samples were collected from healthy people and SARS-COV-2-infected patients, and total RNA was extracted from the nasopharyngeal swab samples as an RNA to be detected;
a conserved zone of an N gene in the SARS-COV-2 was selected as a target binding zone. A sequence is shown in SEQ ID NO. 7:

(SEQ ID NO. 7)
ACCGAAGAGCUACCAGACGAAUUC.

Preparation of a guide RNA: a primer containing a T7 promoter sequence was synthesized. A reverse primer (T7p-R) for a complementary sequence of the T7 promoter: crRNA-N-R is shown in SEQ ID NO. 2; a forward primer (T7p-F) for the complementary sequence of the T7 promoter: crRNA-N-F is shown in SEQ ID NO. 3. Incomplete double-stranded crDNA was made by double-primer annealing and used as a template for in vitro transcription of the guide RNA. A T7 transcriptase was used for catalyzing transcription, and then subjected to an overnight reaction at 37° C. The crRNA was obtained by using a RNA Clean& Concentrator 100 kit for purification and stored at −20° C. or −80° C.

An ssDNA probe sequence (padlock-N) of the N gene is shown in SEQ ID NO. 8:

(SEQ ID NO. 8)
TAGCTCTTCGGTCCCACCTAAACCCTATAGTGAGTCGTATTAATCCCGC
CTACAGAATTCGTCTGG.

The PROTRACTOR isothermal nucleic acid detection method comprises the following steps. First, the ssDNA probe (padlock-N) and the RNA to be detected were added to a reaction system. The reaction system was composed of 0.8 pM FAM and BHQ1 dual-labeled RNA fluorescent probe, 0.5 pM guide RNA, an enzyme mixture (10 U T4 DNA ligase; 10 U T7 RNA polymerase; 0.5 pM LwaCas13a protein), and a PROTRACTOR reaction buffer. The RNA fluorescent probe was 6-FAM-mArArUrGrGrCmAmArArUrGrGrCmA-BHQ1. The PROTRACTOR reaction buffer included 0.5 mM NTPs, 40 mM Tris-HCl, 10 mM MgCl₂, 0.01 mM-10 mM ATP, and 1 mM DTT, and pH of the PROTRACTOR reaction buffer was within a range of 7.0-8.0.

Fluorescence detection: after mixing of the reaction system, the temperature was set at 37° C. in a fluorescence real-time quantitative PCR instrument (7900HT Fast Real-Time PCR), a fluorescence group of the dual-labeled RNA fluorescent probe was FAM, an acquisition interval of a fluorescent signal was 1 min, and the detection time was 30 min.

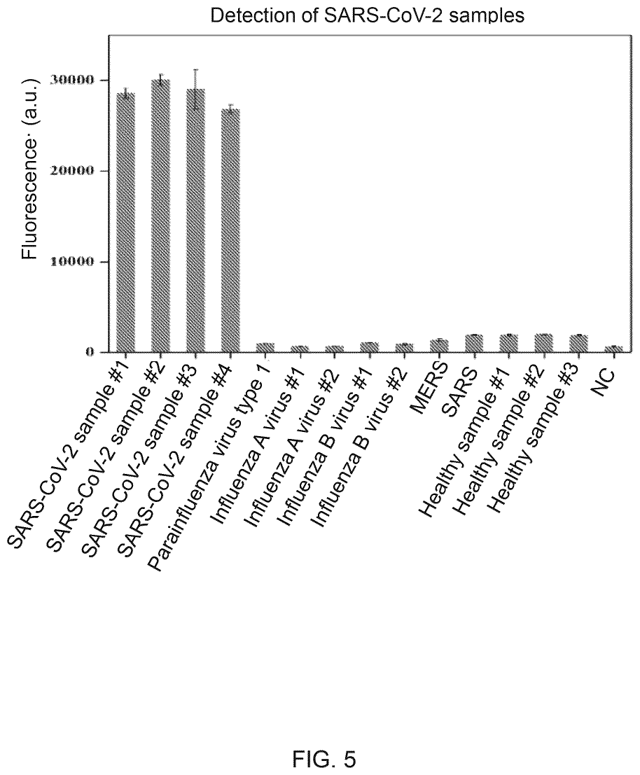

The result of fluorescence detection is shown in FIG. 5. The method can be used for detection of clinical samples of the SARS-COV-2.

### Example 5 a Method for Detecting a Mutation in Genes of SARS-COV-2

This Example detected three single-base mutation sites including an L462R site mutation, a T478K site mutation, and a P681R site mutation in an S gene of the SARS-CoV-2, and differentiated between two SARS-COV-2 subtypes including a W strain and a delta strain using SNP typing.

An original sequence of the W strain (L462R-W) of L462R is shown in SEQ ID NO. 9: AUUAUAAUUACCUGUAUAGAUUGU (SEQ ID NO. 9);
a mutation sequence of the delta strain (L462R-Delta) of L462R is shown in SEQ ID NO. 10: AUUAUAAUUACCGGUAUAGAUUGU (SEQ ID NO. 10);
an original sequence of the W strain (T478K-W) of T478K is shown in SEQ ID NO. 11: AGGCCGGGUAGCACACCUUGUAAUG (SEQ ID NO. 11);
a mutation sequence of the delta strain (T478K-Delta) of T478K is shown in SEQ ID NO. 12: AGGCCGGUAGCAAACCUUGUAAUG (SEQ ID NO. 12);
an original sequence of the W strain (P681R-W) of P681R is shown in SEQ ID NO. 13: AGACUAAUUCUCCUCUCGGGGGGGGCAC (SEQ ID NO. 13);
a mutation sequence of the delta strain (P681R-Delta) of P681R is shown in SEQ ID NO. 14: AGACUAAUUCUCGUCGGGGGGCAC (SEQ ID NO. 14);
preparation of a guide RNA: a primer containing a T7 promoter sequence was synthesized. A reverse primer (T7p-R) for a complementary sequence of the T7 promoter:

crRNA-L452R-R:
(SEQ ID NO. 15)
TTCTTAATACGACTCACTATAGGGATTTAGACTACCCCAAAAACGAAGG
GGACTAA AACACAATCTATACCGGTAATTATAAT;
crRNA-T478K-R:
(SEQ ID NO. 16)
TTCTTAATACGACTCACTATAGGGATTTAGACTACCCCAAAAACGAAGG
GGACTAAAACCATTACAAGGTTTGCTACCGGCCT;
crRNA-P681R-R:
(SEQ ID NO. 17)
TTCTTAATACGACTCACTATAGGGATTTAGACTACCCCAAAAACGAAGG
GGACTAA AACGTGCCCGCCGACGAGAATTAGTCT;

a forward primer (T7p-F) crRNA-F for the complementary sequence of the T7 promoter is shown in SEQ ID NO. 3.

Incomplete double-stranded crDNA was made by double-primer annealing and used as a template for in vitro transcription of the guide RNA. A T7 transcriptase was used for catalyzing transcription, and then subjected to an overnight reaction at 37° C. The crRNA was obtained by using a RNA Clean& Concentrator 100 kit for purification and stored at −20° C. or −80° C.

An L452R typing ssDNA probe sequence (padlock-L452R) of the delta strain of the SARS-COV-2 is shown in SEQ ID NO. 18:

(SEQ ID NO. 18)
GGTAATTATAATCCCAAATCCTCCCTATAGTGAGTCGTATTAATCCCAA
ACAAAACAATCTATAAC.

A T478K typing ssDNA probe sequence (padlock-T478K) of the delta strain of SARS-COV-2 is shown in SEQ ID NO. 19:

(SEQ ID NO. 19)
TGCTACCGGCCTCCCAAACCCACCCTATAGTGAGTCGTATTAATCCCAA
ACAAACATTACAAGGAT.

A P681R typing ssDNA probe sequence (padlock-P681R) of the delta strain of the SARS-COV-2 is shown in SEQ ID NO. 20:

(SEQ ID NO. 20)
GAGAATTAGTCTAACAAACAAACCCTATAGTGAGTCGTATTAATCCCGC
CTACAGTGCCCGCCGCC.

The PROTRACTOR isothermal nucleic acid detection method comprises the following steps. Firstly, an ssDNA probe and the RNA to be detected were added to a reaction system. The reaction system was composed of 0.8 PM FAM and BHQ1 dual-labeled RNA fluorescent probe, 0.5 pM, guide RNA, an enzyme mixture (10 U T4 DNA ligase; 10 U T7 RNA polymerase; 0.5 pM LwaCas13a protein), and a PROTRACTOR reaction buffer. The RNA fluorescent probe was 6-FAM-mArArUrGrGrCmAmArArUrGrGrCmA-BHQ1. The PROTRACTOR reaction buffer included 0.5 mM NTPs, 40 mM Tris-HCl, 10 mM MgCl₂, 0.01 mM-10 mM ATP, and 1 mM DTT, and pH of the PROTRACTOR reaction buffer was within a range of 7.0-8.0.

Fluorescence detection: after mixing of the reaction, the temperature was set at 37° C. in a fluorescence real-time quantitative PCR instrument (7900HT Fast Real-Time PCR), a fluorescence group of the dual-labeled RNA fluorescent probe was FAM, an acquisition interval of a fluorescent signal was 1 min, and the detection time was 30 min.

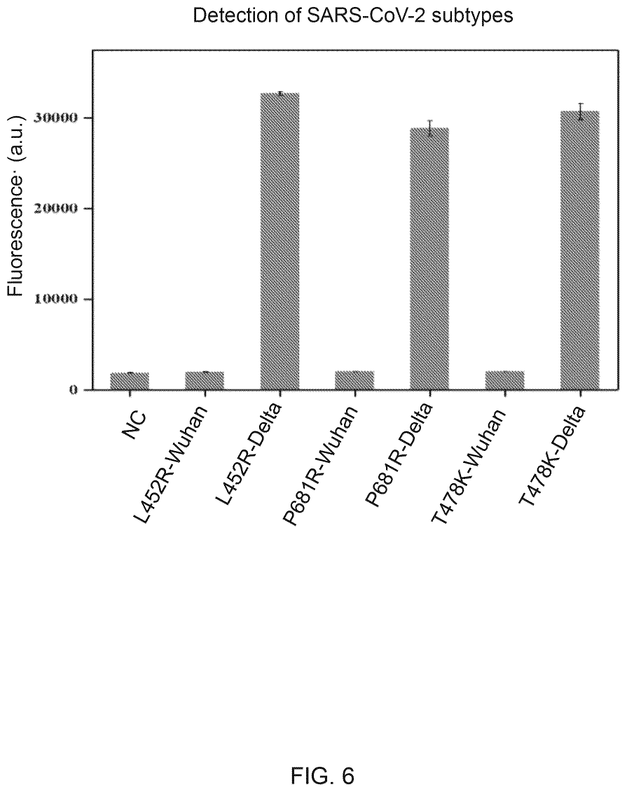

The result of fluorescence detection is shown in FIG. 6. For subtype detection of the SARS-COV-2, the method can differentiate between the two mutation types, the W strain and the delta strain, of the SARS-COV-2 based on the single-base mutation sites.

### Comparative Example 1

The Comparative Example differs from Example 3 in that the PROTRACTOR reaction buffer (B): 0.5 mM NTP mixture, 40 mM Tris-HCl, 10 mM MgCl₂, 10 mM DTT, 0.5 mM ATP, pH 7.5@25° C. of the PROTRACTOR isothermal nucleic acid detection method was replaced with a Buffer 1 (B1): 50 mM Tris-HCl, 10 mM MgCl₂, 10 mM DTT, 0.5 mM ATP, pH 7.5@25° C. used for the stepwise ssDNA cyclization, rolling transcription Buffer 2 (B2): 40 mM Tris-HCl, 6 mM MgCl₂, 10 mM (NH₄)₂SO₄, 1 mM DTT, 2 mM Spermidine, pH 7.9@25° C., and CRISPR/Cas-mediated nucleic acid detection Buffer 3 (B3): 50 mM NaCl, 10 mM Tris-HCl, 10 mM MgCl₂, 100 mg/ml bovine serum protein, pH 7.9@25° C., to verify the advantages of the PROTRACTOR reaction buffer.

Fluorescence detection: after mixing of the reaction, the temperature was set at 37° C. in a fluorescence real-time quantitative PCR instrument (7900HT Fast Real-Time PCR), a fluorescence group of the dual-labeled RNA fluorescent probe was FAM, an acquisition interval of a fluorescent signal was 1 min, and the detection time was 30 min. The RNA fluorescent probe was 6-FAM-mArArUrGrGrCmAmArArUrGrGrCmA-BHQ1.

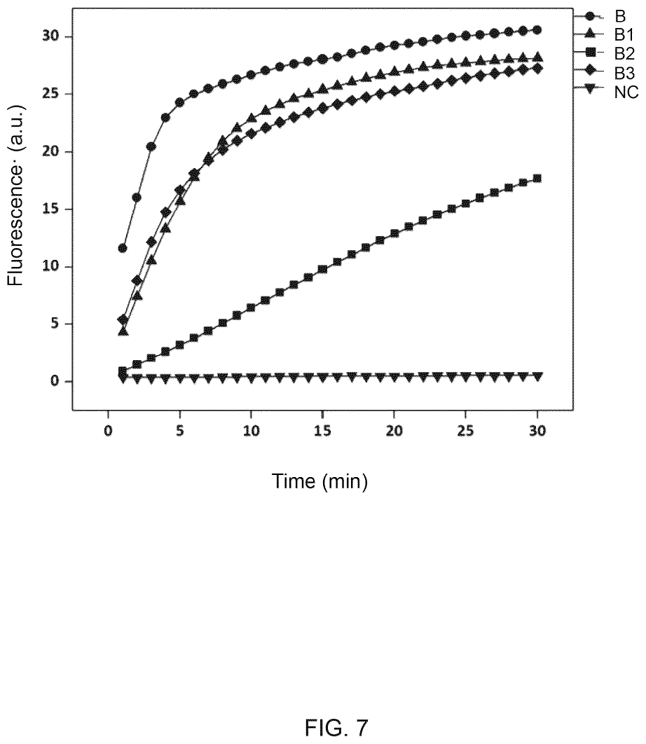

The result of fluorescence detection: as shown in FIG. 7, application of the comparative analysis method indicates that using the PROTRACTOR isothermal nucleic acid detection method in the Example of the present disclosure for detection of the ssRNA with the optimized PROTRACTOR reaction buffer B is better than buffers B1, B2, and B3 in the stepwise detection. The detection time of the PROTRACTOR isothermal nucleic acid detection method in the Example of the present disclosure can be reduced to be within 10 min.

### Comparative Example 2

The Comparative Example differs from Example 3 in that no buffer (NC) was added, and distilled water was used instead of the buffer.

Fluorescence detection: after mixing of the reaction, the temperature was set at 37° C. in a fluorescence real-time quantitative PCR instrument (7900HT Fast Real-Time PCR), a fluorescence group of the dual-labeled RNA fluorescent probe was FAM, an acquisition interval of a fluorescent signal was 1 min, and the detection time was 30 min. The RNA fluorescent probe was 6-FAM-mArArUrGrGrCmAmArArUrGrGrCmA-BHQ1.

The result of fluorescence detection: as shown in FIG. 7, application of the comparative analytical method indicates that the PROTRACTOR isothermal nucleic acid detection method in the Example of the present disclosure is used for detection of the ssRNA, the reaction is essentially not performed without addition of any buffer.

The PROTRACTOR isothermal nucleic acid detection method of the embodiments of the present disclosure comprises ssDNA probe cyclization, transcription, CRISPR-related Cas protein or the variant thereof recognition and shear, and a single-tube one-pot detection reaction. The embodiments of the present disclosure address the difficulty of synchronization of ssDNA probe cyclization, transcription, product recognition and shear in a single reaction system by establishing the PROTRACTOR isothermal nucleic acid detection method for integrating the three steps of reaction. The beneficial effects may be brought by the PROTRACTOR isothermal nucleic acid detection method in the present disclosure include, but are not limited to the following content. Enrichment, amplification, and recognition of the nucleic acid molecules are performed in a single in vitro reaction tube system at room temperature to realize precise, ultra-sensitive and rapid detection of the nucleic acid molecules; the method does not require DNA amplification, PAM sites, or target sequence specific primers, and is adaptable to different types of nucleic acid molecule targets, such as ssDNA, dsDNA and RNA. In particular, for the RNA target detection, no additional step of reverse transcription is required. The PROTRACTOR isothermal nucleic acid detection method of the present disclosure has a wide range of applications in the field of rapid detection of the nucleic acid molecules.

The specific embodiments of the present disclosure are described above. It should be understood that the present disclosure is not limited to the specific embodiments described above, and those skilled in the art may make various deformations or modifications within the scope of the claims, which do not affect the substance of the present disclosure.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure, or feature described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or features may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various parts described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, numbers describing the number of ingredients and attributes are used. It should be understood that such numbers used for the description of the embodiments use the modifier “about”, “approximately”, or “substantially” in some examples. Unless otherwise stated, “about”, “approximately”, or “substantially” indicates that the number is allowed to vary by ±20%. Correspondingly, in some embodiments, the numerical parameters used in the description and claims are approximate values, and the approximate values may be changed according to the required features of individual embodiments. In some embodiments, the numerical parameters should consider the prescribed effective digits and adopt the method of general digit retention. Although the numerical ranges and parameters used to confirm the breadth of the range in some embodiments of the present disclosure are approximate values, in specific embodiments, settings of such numerical values are as accurate as possible within a feasible range.

For each patent, patent application, patent application publication, or other materials cited in the present disclosure, such as articles, books, specifications, publications, documents, or the like, the entire contents of which are hereby incorporated into the present disclosure as a reference. The application history documents that are inconsistent or conflict with the content of the present disclosure are excluded, and the documents that restrict the broadest scope of the claims of the present disclosure (currently or later attached to the present disclosure) are also excluded. It should be noted that if there is any inconsistency or conflict between the description, definition, and/or use of terms in the auxiliary materials of the present disclosure and the content of the present disclosure, the description, definition, and/or use of terms in the present disclosure is subject to the present disclosure.

Finally, it should be understood that the embodiments described in the present disclosure are only used to illustrate the principles of the embodiments of the present disclosure. Other variations may also fall within the scope of the present disclosure. Therefore, as an example and not a limitation, alternative configurations of the embodiments of the present disclosure may be regarded as consistent with the teaching of the present disclosure. Accordingly, the embodiments of the present disclosure are not limited to the embodiments introduced and described in the present disclosure explicitly.

What is claimed is:

1. A PROTRACTOR isothermal nucleic acid detection method, comprising:
S1, extracting total nucleic acid from a sample to be detected;
S2, configuring a reaction system, the reaction system including a single-stranded DNA (ssDNA) probe, an RNA fluorescent probe, a DNA ligase or a variant thereof, an RNA polymerase or a variant thereof, a guide RNA or a derivative thereof, a CRISPR-related Cas protein or a variant thereof, and a PROTRACTOR reaction buffer; wherein the single-ssDNA probe is specifically complementary to a strand of a target nucleic acid molecule;
S3, adding the total nucleic acid extracted in the step S1 to the reaction system of the step S2 to perform thermostatic reaction and generating a fluorescent signal; wherein the ssDNA probe forms a single-stranded circular DNA probe under the action of the DNA ligase or the variant thereof in a process of thermostatic reaction; and
S4, reading and recording the fluorescent signal generated in the step S3, and determining presence or absence of the target nucleic acid molecule in the sample to be detected by the fluorescent signal.

2. The nucleic acid detection method of claim 1, wherein PROTRACTOR is a universal nucleic acid detection platform capable of detecting different types of nucleic acid molecules, including one or more of ssDNA, double-stranded DNA (dsDNA), and single-stranded RNA (ssRNA).

3. The nucleic acid detection method of claim 1, wherein the CRISPR-related Cas protein or the variant thereof is a nuclease having a ssRNA recognition and shear function and a trans-single-stranded RNA shear function; the CRISPR-related Cas protein or the variant thereof includes any one of LbaCas13, LbuC13a, LwaCas13a, AspCas13b, BzoCas13b, CcaCas13b, PsmCas13b, PinCas13b, Pin2Cas13b, Pin3Cas13b, PbuCas13b, PguCas13b, PigCas13b, PsaCas13b, RanCas13b, PspCas13b, EsCas13d, and RspCas13d, or any variant thereof.

4. The nucleic acid detection method of claim 1, wherein the ssDNA probe is composed of a 5′ end arm, a linking sequence, and a 3′ end arm in series; sequences of the 5′ end arm and the 3′ end arm are complementary to one strand of a target nucleic acid molecule sequence; and the linking sequence is a DNA sequence including a complementary sequence of a T7 promoter (T7p).

5. The nucleic acid detection method of claim 1, wherein the RNA fluorescent probe is an ssRNA with a 5′ end labeled with any fluorescence group of FAM, HEX, VIC, Cy5, Cy3, TET, ROX, FITC, and Joe, and a 3′ end labeled with any fluorescence quenching group of TAMRA, BHQ1, MGB, and BHQ2.

6. The nucleic acid detection method of claim 1, wherein the DNA ligase catalyzes formation of a phosphodiester bond between two nucleotide strands using the energy of ATP, and is used to join dsDNA molecules or ssDNA gaps of an RNA/DNA hybrid duplex; wherein the DNA ligase or the variant thereof includes any one of a T4 DNA ligase, an E. coli DNA ligase, a SplintR ligase, and a HiFi Taq DNA ligase, or any variant thereof.

7. The nucleic acid detection method of claim 1, wherein the guide RNA or the derivative thereof is complementary to a sequence of the target nucleic acid molecule.

8. The nucleic acid detection method of claim 1, wherein main components of the PROTRACTOR reaction buffer include 0.1 mM-5 mM NTPs, 10 mM-100 mM Tris-HCl, 0.5 mM-10 mM MgCl₂, 0.01 mM-10 mM ATP, and 0.5 mM-10 mM DTT, pH of the PROTRACTOR reaction buffer being within a range of 6.5-8.0.

9. The nucleic acid detection method of claim 1, wherein the RNA polymerase or the variant thereof is selected from any one of a T7 RNA polymerase, an E. coli RNA polymerase, a T3 RNA polymerase, and an SP6 RNA polymerase, or any variant thereof.


SEQUENCE LISTING (20 sequences)

SEQ ID NO: 1 - DNA, 245 bp, synthetic construct
  source 1..245: mol_type = other DNA
gtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggt

SEQ ID NO: 2 - DNA, 83 bp, synthetic construct
  source 1..83: mol_type = other DNA
ttcttaatacgactcactatagggatttagactaccccaaaaacgaaggggactaaaacggtaattataattaccaccaacct

SEQ ID NO: 3 - DNA, 23 bp, synthetic construct
  source 1..23: mol_type = other DNA
cctatagtgagtcgtattaagaa

SEQ ID NO: 4 - DNA, 66 bp, synthetic construct
  source 1..66: mol_type = other DNA
taccaccaacctccaacctaaaccctatagtgagtcgtattaatcccgcctacaggtaattataat

SEQ ID NO: 5 - DNA, 61 bp, synthetic construct
  source 1..61: mol_type = other DNA
gattctaaggttggtggtaattataattacctgtatagattgtttaggaagtctaatctca

SEQ ID NO: 6 - RNA, 245 nt, synthetic construct
  source 1..245: mol_type = other RNA
gtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggt

SEQ ID NO: 7 - RNA, 24 nt, synthetic construct
  source 1..24: mol_type = other RNA
accgaagagctaccagacgaattc

SEQ ID NO: 8 - DNA, 66 bp, synthetic construct
  source 1..66: mol_type = other DNA
tagctcttcggtcccacctaaaccctatagtgagtcgtattaatcccgcctacagaattcgtctgg

SEQ ID NO: 9 - RNA, 24 nt, synthetic construct
  source 1..24: mol_type = other RNA
attataattacctgtatagattgt

SEQ ID NO: 10 - RNA, 24 nt, synthetic construct
  source 1..24: mol_type = other RNA
attataattaccggtatagattgt

SEQ ID NO: 11 - RNA, 24 nt, synthetic construct
  source 1..24: mol_type = other RNA
aggccggtagcacaccttgtaatg

SEQ ID NO: 12 - RNA, 24 nt, synthetic construct
  source 1..24: mol_type = other RNA
aggccggtagcaaaccttgtaatg

SEQ ID NO: 13 - RNA, 24 nt, synthetic construct
  source 1..24: mol_type = other RNA
agactaattctcctcggcgggcac

SEQ ID NO: 14 - RNA, 24 nt, synthetic construct
  source 1..24: mol_type = other RNA
agactaattctcgtcggcgggcac

SEQ ID NO: 15 - DNA, 83 bp, synthetic construct
  source 1..83: mol_type = other DNA
ttcttaatacgactcactatagggatttagactaccccaaaaacgaaggggactaaaacacaatctataccggtaattataat

SEQ ID NO: 16 - DNA, 83 bp, synthetic construct
  source 1..83: mol_type = other DNA
ttcttaatacgactcactatagggatttagactaccccaaaaacgaaggggactaaaaccattacaaggtttgctaccggcct

SEQ ID NO: 17 - DNA, 83 bp, synthetic construct
  source 1..83: mol_type = other DNA
ttcttaatacgactcactatagggatttagactaccccaaaaacgaaggggactaaaacgtgcccgccgacgagaattagtct

SEQ ID NO: 18 - DNA, 66 bp, synthetic construct
  source 1..66: mol_type = other DNA
ggtaattataatcccaaatcctccctatagtgagtcgtattaatcccaaacaaaacaatctataac

SEQ ID NO: 19 - DNA, 66 bp, synthetic construct
  source 1..66: mol_type = other DNA
tgctaccggcctcccaaacccaccctatagtgagtcgtattaatcccaaacaaacattacaaggat

SEQ ID NO: 20 - DNA, 66 bp, synthetic construct
  source 1..66: mol_type = other DNA
gagaattagtctaacaaacaaaccctatagtgagtcgtattaatcccgcctacagtgcccgccgcc